(19) United States
(12) Patent Application Publication
(10) Pub. No.: US 2024/0102997 A1
(43) Pub. Date: Mar. 28, 2024

(54) Biotin Blocking Methods

(71) Applicants: Marc Key, Ojai, CA (US); Diagnostic BioSystems, Pleasanton, CA (US)

(72) Inventors: Marc Key, Ojai, CA (US); Bipin Gupta, Pleasanton, CA (US)

(73) Assignees: Marc Key, Ojai, CA (US); Diagnostic BioSystems, Pleasanton, CA (US)

(21) Appl. No.: 18/097,366

(22) Filed: Jan. 16, 2023

Related U.S. Application Data
(60) Provisional application No. 63/409,812, filed on Sep. 25, 2022.

(51) Int. Cl. G01N 33/543

(52) U.S. Cl. CPC G01N 33/54306; G01N 33/54393

(57) ABSTRACT

Improved biotin blocking methods are provided which utilize a high-affinity monomeric biotin-binding protein of the avidin family that have only a single biotin binding site. Because the biotin-binding protein is monovalent, it can be applied as a single reagent. Since it does not introduce any additional biotin binding sites, as would be the case with native tetrameric streptavidin, no further blocking of biotin binding sites is necessary. Furthermore, because it is a single reagent, it can be combined with other steps such as the peroxidase blocking step, and both endogenous peroxidase and endogenous biotin can be blocked simultaneously. The biotin blocking methods may be used in the performance of immunoassays, such as tissue sample containing endogenous biotin is stained by IHC to detect a specific analyte. Prior to staining, the tissue is first blocked for endogenous biotin, according to the present invention, by the application of a high-affinity biotin-binding protein.

### CROSS REFERENCE TO RELATED APPLICATIONS

This application claims priority to and the benefit of the filing date of U.S. Provisional Application No. 63/409,812, filed on Sep. 25, 2022, entitled “Biotin Blocking Method”, which is hereby incorporated by reference in its entirety.

### FIELD OF THE INVENTION

This patent specification relates to the field of biological tissue staining. More specifically, this patent specification relates to a method for blocking of nonspecific staining of endogenous biotin in a biological sample.

### BACKGROUND

Biological samples of cells and/or tissues are obtained for purposes of analyzing the biological constituents that comprise the sample. Sample analysis is commonly performed by methods of immunohistochemistry (IHC) staining for analyzing protein components. A commonly used IHC staining method includes the use of the biotin-streptavidin method for detection and a conversion of a substrate/chromogen by peroxidase for staining. An example of this commonly used IHC staining method is provided below which can be broken down into parts, and each part can be further broken down into steps.

The first part of IHC staining involves preparing a biological sample for investigation, such as a tissue suspected of having some disease. An example would be a tissue biopsy of a suspected cancer lesion. The biopsy may then be studied to see if it contains a particular molecular marker, or target of interest. If the target of interest is present in the biopsy, then the diagnosis of cancer is confirmed, whereas if the target of interest is not present in the biopsy, then the diagnosis of cancer may be ruled out. In the IHC staining method, the target of interest is an antigen.

Preparation of a biological sample for histological examination typically involves the following six steps.
1. A tissue sample is obtained and placed into fixative.
2. After fixation the tissue is dehydrated through a series of graded alcohols to remove all water.
3. After dehydration the tissue is placed into a series of baths containing a paraffin solvent such as xylene. The baths contain decreasing concentrations of alcohol and increasing concentrations of xylene. When the tissue reaches the final bath, the alcohol has been completely replaced with xylene.
4. Once the tissue is infiltrated with xylene it can be embedded into paraffin by placing the tissue into heated paraffin that has been melted. The melted paraffin can now infiltrate the tissue replacing the xylene.
5. When cooled the paraffin solidifies to form a solid paraffin block containing the tissue.
6. The paraffin block containing the tissue can be cut into thin slices of approximately 4 micrometers, and some of the cut sections are attached to a microscope slide in preparation for staining and microscopic analysis.

The steps described above for preparation of a biological sample are standard histological methods that precede many different types of histological analysis.

Next, preparation of a biological sample on a microscope slide for IHC staining is performed typically including the following steps:
1. the paraffin is removed from the sample by a process called deparaffinization; and
2. The paraffin is replaced in the tissue with an aqueous solution, called rehydration.

Next, and Antigen Retrieval Procedure is performed which may include the following four steps.
1. A deparaffinized and rehydrated sample on a microscope slide is placed into an antigen retrieval solution. Typical antigen retrieval solutions include 0.05 molar (M) Tris plus, 0.05M EDTA in water at a pH of 9.0, or 0.1M Citric acid solution, pH 6.
2. The antigen retrieval solution with the microscope slides is heated to an elevated temperature of about 90 C-125 C for a sufficient length of time to expose the hidden antigens.
3. The slides are then placed into a buffer bath to cool.
4. The slides with the attached tissues are now ready for IHC staining.

The tissue samples thus prepared, must be blocked for endogenous elements that otherwise would interfere with accurate results. Such endogenous elements would include endogenous enzymes, such as endogenous peroxidase. Because the final staining reaction relies on conversion of a substrate/chromogen by HRP, any endogenous peroxidase must be eliminated to prevent interference by this endogenous enzyme. Typical peroxidase blocking methods include a dilute solution of hydrogen peroxide, hydrogen peroxide in combination with methanol, or hydrogen peroxide in combination with Sodium aside. To block endogenous peroxidase, the tissues are typically incubated with a peroxidase blocking solution for about 5 minutes, or for a time sufficient to block endogenous peroxidase.

Blocking Methods for Endogenous Biotin.

If the TIC staining method relies upon a biotin-streptavidin method for detection, then endogenous biotin must be blocked to prevent unwanted binding of the streptavidin reagent. Biotin is a small vitamin in the B family that is present in many biological samples. Such biotin is identified as endogenous biotin. Furthermore, biotin has a strong affinity for certain biotin-binding proteins, such as avidin and streptavidin. These proteins contain four subunits consisting of a molecular weight of approximately 13,000 Da, each containing a single biotin-binding site. Streptavidin is a tetrameric protein that is widely used as a probe in various molecular detection systems, particularly because of the high affinity of streptavidin for biotin. Avidin and streptavidin can be applied to a tissue containing endogenous biotin, and will bind to the biotin. However, because these proteins are tetravalent, there will be many remaining biotin binding sites. Thus, multivalent biotin binding proteins cannot be used to effectively block endogenous biotin without introducing additional free biotin-binding sites. One technical challenge in the streptavidin-biotin system is the presence of endogenous biotin in many biological samples which can act as an interfering substance.

Typically, a biotin-blocking method requires two steps.
1. In the first step, a multivalent biotin-binding protein is applied to the tissue. Such proteins include avidin and streptavidin. The biotin-binding protein is incubated for about five minutes, or for a sufficient length of time to block endogenous biotin. Because the biotin-binding proteins are multivalent, this step creates numerous biotin-binding sites that were not present before the application of the biotin-binding proteins. Because these biotin-binding sites would bind to any subsequent streptavidin reagent, such as SA-HRP, these sites must now be blocked prior to proceeding with the TIC staining.
2. In the second step, the second reagent in this biotin blocking method is now applied. This step includes the application of free-biotin. Because biotin is monovalent, it will occupy each of the remaining biotin binding sites. Once this step is completed, the residual unbound biotin is rinsed away.

Upon successful completion of this biotin-blocking method, the endogenous biotin sites present in the tissue sample will be neutralized rendering them incapable of reacting with any subsequent streptavidin reagent.

As can be seen by the above description, the two-step biotin blocking method is cumbersome, time-consuming, and expensive. Consequently, a need exists for novel biotin-blocking methods which eliminates one or more steps that are required for existing methods.

### BRIEF SUMMARY OF THE INVENTION

Improved biotin blocking methods are provided which utilize a high-affinity monomeric biotin-binding protein of the avidin family that have only a single biotin binding site. Because the biotin-binding protein is monovalent, it can be applied as a single reagent. Since it does not introduce any additional biotin binding sites, as would be the case with native tetrameric streptavidin, no further blocking of biotin binding sites is necessary. Furthermore, because it is a single reagent, it can be combined with other steps such as the peroxidase blocking step, and both endogenous peroxidase and endogenous biotin can be blocked simultaneously. The biotin blocking methods may be used in the performance of immunoassays, such as tissue sample containing endogenous biotin is stained by IHC to detect a specific analyte. Prior to staining, the tissue is first blocked for endogenous biotin, according to the present invention, by the application of a high-affinity biotin-binding protein.

According to one aspect consistent with the principles of the invention, a biotin blocking method is provided. The method may include the steps of: reacting a tissue sample with a reagent containing a high-affinity monomeric biotin-binding protein; reacting the tissue sample with a primary antibody to an analyte under investigation; reacting the tissue sample with a secondary antibody containing biotin; reacting the tissue sample with a biotin-binding protein that is linked with an enzyme, such as peroxidase or alkaline phosphatase; and reacting the tissue sample with a substrate/chromogen reagent to the enzyme. Preferably, the tissue sample may be simultaneously reacted with the reagent containing a high-affinity monomeric biotin-binding protein and with a peroxidase-blocking reagent. Optionally, the tissue sample may be simultaneously reacted with the reagent containing a high-affinity monomeric biotin-binding protein and with a wash buffer.

According to another aspect, another biotin blocking method is provided. The method may include the steps of: reacting a tissue sample simultaneously with a reagent containing a high-affinity monomeric biotin-binding protein and with a primary antibody to an analyte under investigation; reacting the tissue sample with a secondary antibody containing biotin; reacting the tissue sample with a biotin-binding protein that is linked with a peroxidase; and reacting the tissue sample with a substrate/chromogen reagent to peroxidase.

Numerous objects, features and advantages of the present invention will be readily apparent to those of ordinary skill in the art. One object of the present invention is to provide biotin blocking methods which are able to be used during immunoassays and other reactions which have a decreased number of steps than existing conventional methods.

### BRIEF DESCRIPTION OF THE DRAWINGS

Some embodiments of the present invention are illustrated as an example and are not limited by the figures of the accompanying drawings, in which like references may indicate similar elements and in which:

FIG. 1—FIG. 1 depicts a block diagram of an example of a biotin blocking method according to various embodiments described herein.

FIG. 2—FIG. 2 illustrates a block diagram of another example of a biotin blocking method according to various embodiments described herein.

FIG. 3—FIG. 3 shows a diagram of example results of using the biotin blocking methods of the present invention to block endogenous biotin during immunoassays in which a tissue sample containing endogenous biotin is stained to detect a specific analyte in a tissue sample according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, more preferably within about 5% of the actual desired value and even more preferably within about 1% of the actual desired value of any variable, element or limit set forth herein.

A new biotin blocking method is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

The present invention will now be described by example and through referencing the appended figures representing preferred and alternative embodiments.

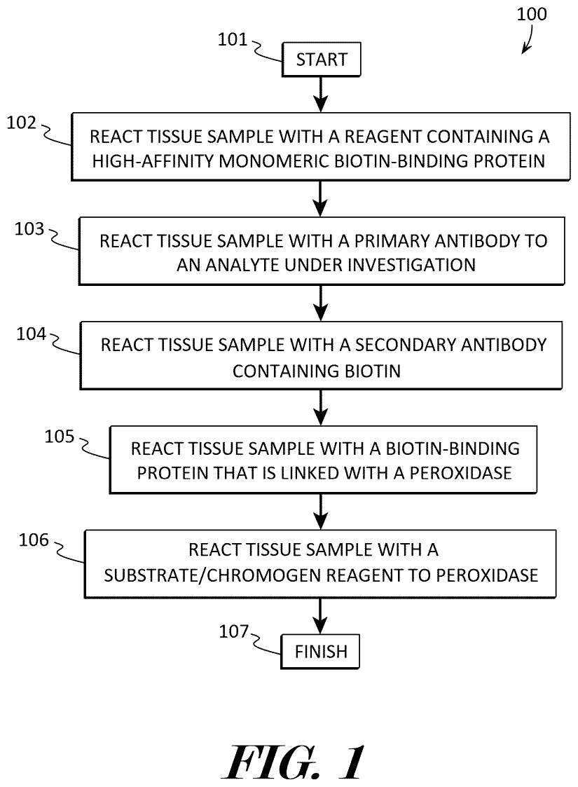

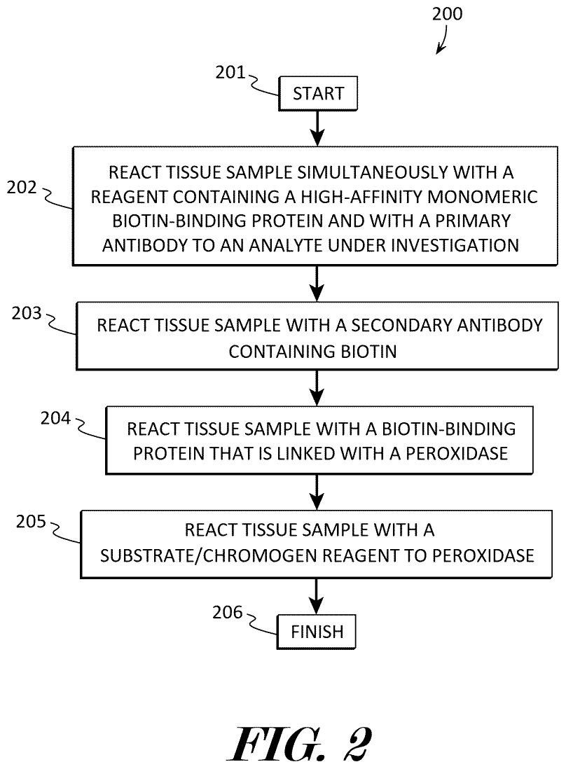

FIG. 1 illustrates an example of a biotin blocking method (“the method”) 100 and FIG. 2 illustrates another example of a biotin blocking method (“the method”) 200 according to various embodiments. In some embodiments, the methods 100, 200, may comprise the use of biotin-binding proteins, such as streptavidin. Streptavidin is a tetrameric protein that is widely used as a probe in various molecular detection systems. It is particularly useful because of its high affinity for biotin. One technical challenge in the streptavidin-biotin system is the presence of endogenous biotin in many biological samples which can act as an interfering substance. In preferred embodiments, the methods 100, 200, may utilize one or more reagents which contain a biotin-binding protein that can be used to bind endogenous biotin in a biological sample. Once bound by the biotin-binding protein, the endogenous biotin is rendered inactive and incapable of any further reactions with subsequent biotin-binding proteins. The inactivation of endogenous biotin can be exploited to eliminate background staining in certain immunoassays such as immunohistochemistry (IHC) and In Situ Hybridization (ISH).

Monovalent Biotin-Binding Protein

Biotin is a small vitamin in the B family that is present in many biological samples. Such biotin is identified as endogenous biotin. Furthermore, biotin has a strong affinity for certain biotin-binding proteins, such as avidin and streptavidin. These proteins contain four subunits consisting of a molecular weight of approximately 13,000 Da, each containing a single biotin-binding site.

Avidin and streptavidin can be applied to a tissue containing endogenous biotin, and will bind to the biotin. However, because these proteins are tetravalent, there will be many remaining biotin binding sites. Thus, multivalent biotin binding proteins cannot be used to effectively block endogenous biotin without introducing additional free biotin-binding sites.

Biotin-binding proteins may be used in certain immunoassays to suppress background staining. Typically, these immunoassays use biotin and a biotin-binding avidin protein, such as avidin or streptavidin, as part of their signal-generating system. Numerous avidin-encoding genes have been identified in the phyla Actinobacteria and Proteobacteria. The diversity of protein sequences is high and several new variants of genes encoding biotin-binding avidins have been described in the scientific literature. In order to better understand the role of biotin and biotin-binding avidin proteins in these systems the following steps would represent a typical immunoassay:
1. A biological sample is reacted with a primary antibody. If the target analyte is present in the biological sample the antibody will bind to the analyte. The primary antibody is further labeled with biotin using standard conjugation methods.
2. A second reagent containing a biotin-binding avidin protein, such as streptavidin, is added to the biological sample. If an antibody-binding event has occurred the biotin-binding avidin protein will bind to the biotin. Furthermore, the biotin-binding avidin protein is labeled with peroxidase enzyme using standard conjugation methods.
3. The complex containing peroxidase is next reacted with a peroxidase substrate to yield a colored reaction product. The colored reaction product can be observed and measured. A colored reaction product is indicative of the presence of the analyte whereas a colorless reaction is indicative of the absence of the analyte.

In preferred embodiments, the methods 100, 200, may be used to perform immunoassays, such as immunohistochemistry (IHC) and immunocytochemistry (ICC). Immunohistochemistry is an immunoassay in which the biological sample is a tissue, and immunocytochemistry is an immunoassay in which the biological sample is a collection of cells. Simply put, IHC is performed on samples derived from tissues that have been histologically processed into thin sections and the staining process exploits enzymes which catalyze the deposition of a colored staining product at antigenic sites within the sample. ICC relies on the same enzyme reactions as IHC, but it is performed on samples consisting of cells grown in a monolayer or cells in suspension which are deposited on a slide.

Methods of immunoassay may rely on reagents containing biotin and streptavidin. Below are the typical steps of a biotin-streptavidin IHC method.
1. A tissue sample is reacted with a primary antibody, usually derived from a mouse or rabbit host, and directed against a particular analyte under investigation. The tissue is incubated for a sufficient time to allow the primary antibody to bind to its analyte, if present.
2. The tissue sample is then reacted with a secondary antibody that will bind to the primary antibody. For example, the secondary antibody may be an antibody that reacts with mouse antibodies or the secondary antibody may be an antibody that reacts with rabbit antibodies. Furthermore, the secondary antibody is labeled with biotin.
3. The tissue sample is next reacted with streptavidin conjugated to Horseradish peroxidase (SA-HRP). The SA-HRP will bind to the biotin of the preceding step, if present.
4. The last step involves incubation of the tissue sample with a peroxidase substrate. If SA-RP is present an insoluble colored chromogen will be deposited on the tissue sample, which can then be viewed microscopically.
5. The presence of a colored reaction product is indicative of a positive test for the analyte, whereas the absence of a colored reaction product is indicative of a negative test for the analyte.

Avidin is a protein isolated from eggs with biotin-binding properties. Another similar protein is streptavidin that is isolated from the bacterium Streptomyces avidinii. Both avidin and streptavidin are tetramers, having four identical subunits. Each subunit can bind a single molecule of biotin. Because of their tetrameric structure, these proteins cannot be used alone to create an effective biotin block in immunoassays. If avidin or streptavidin is used to block endogenous biotin these proteins will bind to the endogenous biotin molecule via one of the tetramers, but at the same time the other three tetramers are capable of binding to subsequent biotin. Since these immunoassays rely on subsequent biotin binding reactions, this type of biotin-blocking method with tetrameric avidin or streptavidin will not work.

Monomeric Biotin-Binding Avidin Proteins

Monomeric streptavidin has been generated by reduction of tetrameric streptavidin to its individual subunits. However, these forms of monomeric streptavidin have been shown to have a low affinity for biotin and are not useful reagents in immunoassays. Monomeric streptavidin has also been generated by genetic engineering to produce a monomeric protein of approximately 13-16 KDa. However, most of these monomers have generally shown low affinity for biotin. Monomeric streptavidin, monomeric avidin, and other monomeric biotin-binding avidin proteins having a low affinity for binding biotin

Monomeric biotin-binding avidin proteins generated by reduction of tetrameric biotin-binding avidin proteins and Monomeric biotin-binding avidin proteins generated by genetic engineering to produce a monomeric protein of approximately 13-16 KDa may be referred to herein as low-affinity monomeric biotin-binding proteins. Generally, these low-affinity monomeric biotin-binding proteins may comprise a biotin dissociation constant (KD) that is greater than 10.0 Nanomolar (nM), such as 10.1 nM to 100 nM. Generally, these low-affinity monomeric biotin-binding proteins may comprise a biotin rate constant of dissociation (Koff) that is greater than 10.0×10⁻³ minutes⁻¹, such as 10.1×10⁻³ minutes⁻¹ to 100×10⁻³ minutes⁻¹. These low-affinity monomeric biotin-binding proteins may comprise a biotin binding half-life (t½) of between 1 minute to less than 100 minutes.

On the other hand, the methods 100, 200, of the present invention use a high-affinity monomeric biotin-binding protein, such as monomeric streptavidin and monovalent avidin, to develop an effective biotin-blocking system. These high-affinity monomeric biotin-binding proteins may be added to a biological sample to bind endogenous biotin. Since these proteins are monomeric and monovalent, after the biotin-binding event has occurred both the endogenous biotin and the monovalent biotin-binding proteins are rendered inactive and cannot interfere with any subsequent steps that utilize biotin, avidin, or streptavidin as part of their signal-generating system. These high-affinity monomeric biotin-binding proteins may comprise a biotin dissociation constant (KD) that is less than 10.0 Nanomolar (nM), such as between 1.0 nM and 10.0 nM. These high-affinity monomeric biotin-binding proteins may comprise a biotin rate constant of dissociation (Koff) that is less than 10.0×10⁻³ minutes⁻¹, such as between 1.0×10⁻³ minutes⁻¹ and 10.0×10⁻³ minutes⁻¹. These high-affinity monomeric biotin-binding proteins may comprise a biotin binding half-life (t½) of between 100 to over 300 minutes. In preferred embodiments, the high-affinity monomeric biotin-binding proteins are based on avidin and streptavidin. Other biotin-binding proteins have been described, but have not been widely used in immunoassays because of their lower affinity for biotin. In further preferred embodiments, high-affinity monomeric biotin-binding proteins used by the method 100 of the present invention have been genetically engineered, and their binding affinities for biotin are similar to the native tetrameric structures, making them ideal reagents for binding biotin, or in this case for preparing biotin-blocking reagents. Likewise, any monovalent or monomeric high affinity biotin-blocking molecule, whether or not related to avidin or streptavidin, may be used by the method 100 achieve the same effect of blocking the endogenous biotin.

In preferred embodiments, a high-affinity monomeric biotin-binding protein may comprise a high-affinity monovalent streptavidin. In further preferred embodiments, a high-affinity monovalent streptavidin may comprise the genetically engineered monomeric streptavidin that is commercially available by the name of SAvPhire™ Monomeric Streptavidin (produced by Sigma-Aldridge, St. Louis, MO). This high-affinity monomeric streptavidin was shown to be monovalent for biotin, and had a high binding affinity for biotin similar to native streptavidin. In preferred embodiments, high-affinity monomeric streptavidin may comprise SAvPhire™ Monomeric Streptavidin.

As can be seen by the above description, the presence of any endogenous biotin in an immunoassay tissue sample will bind to the SA-RP causing a false positive test. Therefore, any endogenous biotin must be blocked prior to performing the immunoassay. Certain biotin blocking methods have been previously proposed, but these methods are cumbersome and require multiple steps. These previous methods will be described briefly.

Avidin and streptavidin are tetrameric structures and can bind four biotin molecules. If these proteins are used to bind to, and block, endogenous biotin, the following reaction occurs:
1. One of the tetramer arms binds to the endogenous biotin in the tissue, thereby eliminating endogenous biotin as a background generating element, but at the same time introducing three additional biotin-binding sites, one on each of the three remaining arms. These biotin-binding sites can now bind the secondary biotinylated antibody, even in the absence of any analyte-antibody reaction. This method of blocking is ineffective because it simply trades one type of background, caused by biotin, for another type of background, caused by streptavidin.

The methods 100, 200, of the present invention provide simple and effective ways of blocking endogenous biotin without introducing any other unwanted sources of background. In preferred embodiments, the methods 100, 200, may comprise the step of reacting a tissue sample with a reagent containing a high-affinity monomeric biotin-binding protein, such as SAvPhire™ Monomeric Streptavidin. The advantage of these methods 100, 200, can be appreciated by understanding that once the high-affinity monomeric biotin-binding protein has bound to endogenous biotin, there are no other biotin-binding sites available and the endogenous biotin and the high-affinity monomeric biotin-binding protein have effectively neutralized each other, so that neither can participate in any subsequent biotin and biotin-binding avidin protein interactions.

Referring to FIG. 1, a block diagram of a first example biotin blocking method (“the method”) 100 is provided. This improved biotin-blocking method preferably utilizes a high-affinity monomeric biotin-binding protein of the avidin family that has only a single biotin binding site. Because the high-affinity monomeric biotin-binding protein is monovalent, it can be applied as a single reagent. Since it does not introduce any additional biotin binding sites, as would be the case with native tetrameric streptavidin, no further blocking of biotin binding sites is necessary. Furthermore, because it is a single reagent, it can be combined with other steps such as the peroxidase blocking step, and both endogenous peroxidase and endogenous biotin can be blocked simultaneously. The method 100 may be used in the performance of immunoassays, such as tissue sample containing endogenous biotin is stained by IHC to detect a specific analyte. Prior to staining, the tissue is first blocked for endogenous biotin, according to the present invention, by the application of a high-affinity biotin-binding protein. The high-affinity biotin binding protein may be monovalent, such that each protein molecule binds only a single biotin molecule present in the tissue.

In some embodiments, the method 100 may start 101 and a tissue sample may be reacted with a reagent containing a monomeric biotin-binding protein in step 102. Any endogenous biotin present in the tissue sample will be bound by the protein and rendered inactive. In preferred embodiments, the high-affinity monomeric biotin-binding protein may comprise a high-affinity monovalent avidin, a high-affinity monovalent streptavidin, and/or a high-affinity monovalent biotin binding protein of the avidin family. In further preferred embodiments, the high-affinity monomeric biotin-binding protein comprises a biotin dissociation constant (KD) that is less than 10.0 Nanomolar (nM). In still further preferred embodiments, the high-affinity monomeric biotin-binding protein comprises a biotin rate constant of dissociation (Koff) that is less than 10.0×10⁻³ minutes⁻¹.

In some embodiments, the tissue sample may be reacted with a reagent containing a monomeric biotin-binding protein may be applied as an incubation step prior to the addition of any subsequent streptavidin reagent, such as SA-HRP. The IHC steps prior to SA-HRP include 1) a blocking step for endogenous enzymes, and 2) the application of the primary antibody. If the biotin-blocking step can be combined with one of these steps, then the overall number of steps required for IHC staining (and other similar immunoassays) would be reduced by one. Referring to Example 7 below, a high-affinity monomeric biotin-binding protein was mixed with the peroxidase inhibition step. The results showed that both endogenous biotin and endogenous peroxidase were inactivated by this single blocking reagent.

Combining Monomeric Biotin-Binding Proteins with a Peroxidase Inhibitor

Many immunoassays rely upon an enzyme as part of their signal-generating system. A commonly used enzyme is peroxidase, such as Horseradish Peroxidase (HRP). Biological samples frequently contain endogenous peroxidase enzymes or peroxidase-like enzymes such as catalase. These endogenous enzymes must be inactivated; otherwise, a false-positive signal may occur. Several peroxidase blocking methods have been developed. These enzyme-blocking reagents are generally applied to the biological sample as a first step in order to block any endogenous enzyme activity prior to commencing with the immunoassay.

In preferred embodiments, in step 102, the tissue sample may be simultaneously reacted with a reagent containing a high-affinity monomeric biotin-binding protein and a peroxidase-blocking reagent, such as a reagent that comprises hydrogen peroxide (H₂O₂). In further embodiments, the tissue sample may be simultaneously reacted with a reagent containing a high-affinity monomeric biotin-binding protein and with two or more, such as a plurality of these peroxidase-blocking reagents, thereby creating a dual blocking reagent that can block both endogenous biotin and endogenous peroxidase-like activity with a single reagent.

In some embodiments, in step 102, the high-affinity biotin-binding protein may be contained in a wash buffer, so that the tissue sample may be simultaneously subjected to a reagent containing a high-affinity monomeric biotin-binding protein and a wash buffer, e.g., a 0.05M Tris-HCl buffer, pH 7.5, or a 0.05M phosphate buffer, pH 7.5. As tissue samples are rinsed in a wash buffer between each immunoassay step, the incorporation of the high-affinity monomeric biotin-binding protein in a wash buffer would achieve the same effect of blocking endogenous biotin as reacting the sample with a reagent containing a high-affinity monomeric biotin-binding protein alone.

In step 103, the tissue sample may be reacted with a primary antibody to the analyte under investigation. The tissue may be reacted with a primary antibody directed against the analyte of interest, such as the analyte of interest in an immunoassay, such as immunohistochemistry (IHC) or In Situ Hybridization (ISH).

In step 104, the tissue sample is reacted with a secondary antibody containing biotin. Since the biotin-binding protein of step 102 is monomeric, it cannot react with the biotin of the secondary antibody. A biotinylated secondary antibody, directed against the primary antibody, is reacted with the tissue sample in step 104. If the primary antibody has reacted with its analyte and is present in the tissue, then the secondary antibody will react with the primary antibody, thereby incorporating biotin in the analyte/antibody complex. Since the endogenous biotin of the tissue sample has been inactivated, the only active biotin is that carried by the secondary antibody.

In step 105, the tissue sample may be reacted with a biotin-binding protein that is linked with an enzyme, such as an alkaline phosphatase e.g., streptavidin-alkaline phosphatase, a peroxidase e.g., streptavidin-Horseradish peroxidase (SA-HRP), etc. Since the monomeric biotin-binding protein of step 102 has inactivated all the endogenous biotin, the SA biotin-binding protein that is linked with the enzyme cannot react with endogenous biotin. As an example, a streptavidin-HRP reagent is then added to the tissue sample. If biotin is present in the tissue sample, the streptavidin-HRP will bind, thereby incorporating the peroxidase enzyme into the complex. Since the only active biotin is that incorporated into the secondary antibody, no background staining from endogenous biotin will occur.

In step 106, the tissue sample may be reacted with a substrate/chromogen reagent to the enzyme. A colored reaction product is formed if the tissue sample contained the analyte under investigation, whereas no colored reaction product indicates the analyte was absent. Preferably, the tissue sample may be incubated with an enzyme substrate/chromogen that generates a colored reaction product if biotin-binding protein that is linked with the enzyme, e.g., a phosphatase, such as streptavidin-alkaline phosphatase, or a peroxidase, such as Horseradish peroxidase (SA-HRP), is present. Each step of the immunoassay sequence is dependent upon the preceding step. If any step does not complete successfully, no staining will occur. Therefore, if the analyte of interest is absent from the tissue sample the immunoassay sequence will not commence, and no staining will occur. Once the tissue sample is reacted with a substrate/chromogen reagent to the enzyme, the tissue sample may be viewed microscopically to determent whether or not the chromogen is present. After step 106, the method 100 may finish.

Referring to FIG. 2, a block diagram of a second example biotin blocking method (“the method”) 200 is provided. Similar to the method 100 of FIG. 1, this improved biotin-blocking method 200 utilizes a high-affinity monomeric biotin-binding protein of the avidin family that have only a single biotin binding site. Because it is monovalent, it can be applied as a single reagent. Since it does not introduce any additional biotin binding sites, as would be the case with native tetrameric streptavidin, no further blocking of biotin binding sites is necessary. Furthermore, because it is a single reagent, it can be combined with other steps such as the peroxidase blocking step, and both endogenous peroxidase and endogenous biotin can be blocked simultaneously. The method 200 may be used in the performance of immunoassays, such as tissue sample containing endogenous biotin is stained by IHC to detect a specific analyte. Prior to staining, the tissue is first blocked for endogenous biotin, according to the present invention, by the application of a high-affinity biotin-binding protein. The high-affinity biotin binding protein may be monovalent, such that each protein molecule binds only a single biotin molecule present in the tissue.

In some embodiments, the method 200 may start 201 and a tissue sample may be simultaneously reacted with a reagent containing a monomeric biotin-binding protein and with a primary antibody to an analyte under investigation in step 202, such as the analyte of interest in an immunohistochemistry (IHC) assay, an In Situ Hybridization (ISH) assay, etc.

Any endogenous biotin present in the tissue sample will be bound by the protein and rendered inactive while tissue sample may also be reacted with a primary antibody, usually derived from a mouse or rabbit host, and directed against a particular analyte under investigation. The tissue is incubated for a sufficient time to allow the primary antibody to bind to its analyte, if present. Referring to Example 6, a high-affinity monomeric biotin-binding protein was added to the primary antibody and then applied as a single reagent. Subsequent development of the IHC signal showed that the combination of high-affinity monomeric biotin-binding protein and primary antibody inhibits endogenous biotin and had no effect on antibody binding.

In preferred embodiments, the high-affinity monomeric biotin-binding protein may comprise a high-affinity monovalent avidin, a high-affinity monovalent streptavidin, and/or a high-affinity monovalent biotin binding protein of the avidin family. In further preferred embodiments, the high-affinity monomeric biotin-binding protein comprises a biotin dissociation constant (KD) that is less than 10.0 Nanomolar (nM). In still further preferred embodiments, the high-affinity monomeric biotin-binding protein comprises a biotin rate constant of dissociation (Koff) that is less than 10.0×10-3 minutes-1.

In preferred embodiments, in step 202, the tissue sample may also be simultaneously reacted with a reagent containing a high-affinity monomeric biotin-binding protein, and with a peroxidase-blocking reagent, such as a reagent that comprises hydrogen peroxide (H₂O₂). In further embodiments, the tissue sample may be simultaneously reacted with a reagent containing a high-affinity monomeric biotin-binding protein and with two or more, such as a plurality of these peroxidase-blocking reagents, thereby creating a dual blocking reagent that can block both endogenous biotin and endogenous peroxidase-like activity with a single reagent.

In some embodiments, in step 202, the high-affinity biotin-binding protein may also be mixed with wash buffer, so that the tissue sample may be simultaneously subjected to a reagent containing a high-affinity monomeric biotin-binding protein, the primary antibody, and a wash buffer. As tissue samples are rinsed in a wash buffer between each immunoassay step, the incorporation of the high-affinity monomeric biotin-binding protein in a wash buffer would achieve the same effect of blocking endogenous biotin as reacting the sample with a reagent containing a high-affinity monomeric biotin-binding protein alone.

In step 203, the tissue sample is reacted with a secondary antibody containing biotin. Since the biotin-binding protein of step 202 is monomeric, it cannot react with the biotin of the secondary antibody. A biotinylated secondary antibody, directed against the primary antibody, is reacted with the tissue sample in step 203. If the primary antibody has reacted with its analyte and is present in the tissue, then the secondary antibody will react with the primary antibody, thereby incorporating biotin in the analyte/antibody complex. Since the endogenous biotin of the tissue sample has been inactivated, the only active biotin is that carried by the secondary antibody.

In step 204, the tissue sample may be reacted with a biotin-binding protein that is linked with an enzyme, such as an alkaline phosphatase, e.g., streptavidin-alkaline phosphatase, a peroxidase, e.g., streptavidin-Horseradish peroxidase (SA-RP), etc. Since the monomeric biotin-binding protein of step 102 has inactivated all the endogenous biotin, the SA biotin-binding protein that is linked with the enzyme cannot react with endogenous biotin. As an example, a streptavidin-RP reagent is then added to the tissue sample. If biotin is present in the tissue sample, the streptavidin-HRP will bind, thereby incorporating the peroxidase enzyme into the complex. Since the only active biotin is that incorporated into the secondary antibody, no background staining from endogenous biotin will occur.

In step 205, the tissue sample may be reacted with a substrate/chromogen reagent to the enzyme. A colored reaction product is formed if the tissue sample contained the analyte under investigation, whereas no colored reaction product indicates the analyte was absent. Preferably, the tissue sample may be incubated with an enzyme substrate/chromogen that generates a colored reaction product if biotin-binding protein that is linked with the enzyme, e.g., a phosphatase, such as streptavidin-alkaline phosphatase, or a peroxidase, such as Horseradish peroxidase (SA-HRP), is present. Each step of the immunoassay sequence is dependent upon the preceding step. If any step does not complete successfully, no staining will occur. Therefore, if the analyte of interest is absent from the tissue sample the immunoassay sequence will not commence, and no staining will occur. Once the tissue sample is reacted with a substrate/chromogen reagent to the enzyme, the tissue sample may be viewed microscopically to determent whether or not the chromogen is present. After step 205, the method 200 may finish 206.

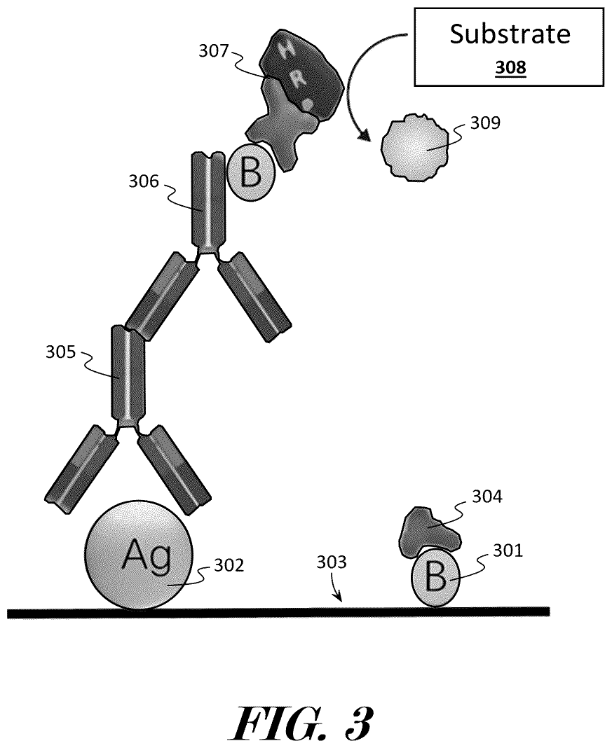

Turning now to FIG. 3, a diagram of the results of using methods 100, 200, to block endogenous biotin during immunoassays in which a tissue sample containing endogenous biotin 301 is stained to detect a specific analyte 302 in a tissue sample 303 is shown. Prior to staining, the tissue 303 is first blocked for endogenous biotin 301, according to the present invention, by the application of a high-affinity monomeric biotin-binding protein 304. The high-affinity monomeric biotin-binding protein 304 may be monovalent, such that each molecule of the high-affinity monomeric biotin-binding protein 304 binds only a single biotin molecule present in the tissue 303. The tissue 303 is next reacted with a primary antibody 305 directed against the analyte 302 of interest. A biotinylated secondary antibody 306, directed against the primary antibody 305, is next reacted with the tissue sample 303. If the primary antibody 305 has reacted with its analyte 302 and is present in the tissue 303, then the biotinylated secondary antibody 306 will react with the primary antibody 305, thereby incorporating biotin in the analyte/antibody complex. Since the endogenous biotin 301 of the tissue sample 303 has been inactivated, the only active biotin is that carried by the secondary antibody 306. A biotin-binding protein that is linked with a peroxidase 307, such as tetravalent streptavidin-HRP reagent, is then added to or reacted with the tissue sample 303. If biotin is present in the analyte/antibody complex, the biotin-binding protein that is linked with a peroxidase 307 will bind, thereby incorporating the peroxidase enzyme into the complex. Since the only active biotin is that which is incorporated into the secondary antibody 306, no background staining from endogenous biotin 301 will occur. Finally, the tissue 303 is incubated or reacted with a peroxidase substrate/chromogen 308 that generates a colored reaction product 309 if the biotin-binding protein that is linked with a peroxidase 307, such as tetravalent streptavidin-HRP reagent, is present.

Referring to Table 1, the methods 100, 200, of the present invention may provide the same immunoassay results, while requiring fewer steps that existing or conventional methods.

TABLE 1
Comparison of Biotin Blocking Methods.

| | Conventional Two-Step | |
|---|---|---|
| Step | Method | Improved Method 100 |
| Peroxidase Block | Yes | No (included in Biotin |
| | | Block #1) |
| Biotin Block #1 | Yes | Yes |
| Biotin Block #2 | Yes | No |

### Example 1. Preparation of a Tissue Sample for IHC Staining

A biological sample containing a tissue is prepared for IHC staining using conventional histochemical methods, including fixation, embedding into paraffin, cutting a thin section of tissue and affixing the tissue section to a glass microscope slide. The microscope slide containing the tissue section is then deparaffinized and rehydrated into a suitable buffer solution. The tissue sample is then prepared for IHC staining by exposing the tissue section to an antigen retrieval solution composed of 0.01M Citrate buffer at pH 6.0. The tissue section, submerged in the antigen retrieval solution is then heated in a pressure cooker to a temperature of 121 C for 15 minutes. The antigen retrieval solution is allowed to cool to about 100 C, and then the tissue sections are removed and placed into a buffer bath. The microscope slides and attached tissue sections, thus prepared, are now ready for IHC staining.

### Example 2. Presence of Endogenous Biotin

Tissue sections were prepared for IHC staining according to the method of Example 1. Tissues were screened for the presence of endogenous biotin by the following steps:
Step 1: Tissues were incubated for five minutes with a solution of streptavidin-peroxidase (Jackson Immunoresearch, West Grove, PA) at a concentration of 10 ug/ml in a Tris-Buffered Saline solution, pH 7.6 (TBS). The streptavidin-peroxidase would bind to any endogenous biotin, if preset.
Step 2: After incubation, slides were rinsed in TBS to remove any unbound streptavidin-peroxidase
Step 3: Slides were incubated with a peroxidase substrate-chromogen solution comprised of 3, 3′-Diaminobenzidine tetrahydrochloride (DAB; Sigma Chemicals) prepared by adding DAB to TBS at a concentration of 1.0 mg/ml. Hydrogen peroxide was added to yield a final concentration of 0.03%. Slides were incubated in the DAB solution for five minutes. The DAB formed a brown reaction product at sites of peroxidase activity.
Step 4: Following incubation, the slides were rinsed in TBS to remove any unbound DAB.
Step 5: Slides were then examined microscopically to identify sites containing brown deposit of DAB chromogen. A positive brown stain was indicative of the presence of endogenous biotin.

Note: Slides may also show staining due to the presence of endogenous peroxidase activity. This activity is mostly seen in the red blood cells (RBC's) and can be easily distinguished from endogenous biotin staining. Unless otherwise specifically noted, as in Example 9, the endogenous peroxidase staining was disregarded, and only endogenous biotin activity was considered.

### Example 3. Screening for Endogenous Biotin in Kidney and Liver Tissues

Tissue samples screened for endogenous biotin, according to Example 2 included: tonsil, placenta, prostate, skin, striated muscle, kidney, and liver. Kidney and liver tissues showed the highest level of endogenous biotin and were selected for subsequent experiments.

### Example 4. Two-Step Method for Blocking of Endogenous Biotin Using Native Streptavidin

Native streptavidin is composed of four identical subunits and is tetravalent with respect to binding biotin. Streptavidin can be used as an endogenous biotin blocking method in combination with a second incubation with biotin to block excess biotin binding sites.

Experimental Procedure:
Step 1: Tissues were incubated with a solution of native streptavidin at a concentration of 1 microgram per milliliter (ug/ml) in TBS for five minutes to allow binding to endogenous biotin.
Step 2: Tissues were rinsed in TBS and then incubated for five minutes with a solution of biotin at a concentration of 0.1 ug/ml for five minutes to allow binding to excess biotin-binding sites.
Step 3: Tissues were rinsed and then incubated for 10 minutes with a biotinylated secondary antibody of goat-antimouse immunoglobulin.
Step 4: Tissues were rinsed and then incubated for 10 minutes with a solution of SA-HRP in TBS at a concentration of 10 ug/ml.
Step 5: Tissues were rinsed and then incubated for five minutes with a solution of DAB.

TABLE 2
Example 4 Group Treatment

| | Step 1 Block | Step 2 Block | Biotinylated | SA- | |
|---|---|---|---|---|---|
| Group | SA | Biotin | 2nd Ab | HRP | DAB |
| 1 | No | No | No | Yes | Yes |
| 2 | No | Yes | Yes | Yes | Yes |
| 3 | Yes | No | Yes | Yes | Yes |
| 4 | Yes | Yes | Yes | Yes | Yes |

TABLE 3
Example 4 Results

| Group | Staining | Interpretation |
|---|---|---|
| 1 | Yes | Endogenous biotin |
| 2 | Yes | Endogenous biotin |
| 3 | Yes | Binding of 2nd Ab to tetrameric streptavidin |
| 4 | No | Inactivation of endogenous biotin |

### Example 5. Endogenous Biotin Blocking with High-Affinity Monovalent Streptavidin

Experimental Procedure:
Step 1: Tissues were incubated with a solution of high-affinity monovalent streptavidin at a concentration of 1.0 ug/ml in TBS for five minutes to allow binding to endogenous biotin.
Step 2: Tissues were rinsed and then incubated for 10 minutes with a biotinylated secondary antibody of goat-antimouse immunoglobulin.
Step 3: Tissues were rinsed and then incubated for 10 minutes with a solution of SA-HRP in TBS at a concentration of 10.0 ug/ml.
Step 4: Tissues were rinsed and then incubated for five minutes with a solution of DAB.

TABLE 4
Example 5 Group Treatment

| | Monomeric | Biotinylated | | |
|---|---|---|---|---|
| Group | SA | 2nd Ab | SA-HRP | DAB |
| 1 | No | No | Yes | Yes |
| 2 | No | Yes | Yes | Yes |
| 3 | Yes | Yes | Yes | Yes |

TABLE 5
Example 5 Results

| Group | Staining | Interpretation |
|---|---|---|
| 1 | Yes | Endogenous biotin |
| 2 | Yes | Endogenous biotin |
| 3 | No | Inactivation of endogenous biotin |

### Example 6. Combination of High-Affinity Monovalent Streptavidin with Antibody Diluent

An Antibody Diluent (ABD) was prepared comprised of 1% Bovine Serum Albumin (BSA) is TBS. In some cases, the ABD included 1.0 ug/ml of high-affinity monovalent streptavidin (monomeric SA). In some cases, the ABD included the primary antibody to cytokeratin (AE1/AE3).

Experimental Procedure:
Step 1: Tissues were incubated with a solution of ABD for five minutes.
Step 2: Tissues were rinsed and then incubated for 10 minutes with a biotinylated secondary antibody of goat-antimouse immunoglobulin.
Step 3: Tissues were rinsed and then incubated for 10 minutes with a solution of SA-HRP in TBS at a concentration of 10.0 ug/ml.
Step 4: Tissues were rinsed and then incubated for five minutes with a solution of DAB.

TABLE 6
Example 6 Group Treatment

| | | Biotinylated | SA- | |
|---|---|---|---|---|
| Group | ABD | Second AB | HRP | DAB |
| 1 | ABD only | Yes | Yes | Yes |
| 2 | ABD + monomeric SA | Yes | Yes | Yes |
| 3 | ABD + monomeric SA + CK | Yes | Yes | Yes |

TABLE 7
Example 6 Results

| Group | Staining | Interpretation |
|---|---|---|
| 1 | Yes | Endogenous biotin |
| 2 | No | Inactivation of endogenous biotin |
| 3 | Yes | Specific staining of CK, Inactivation of endogenous |
| | | biotin |

### Example 7. Combination of High-Affinity Monovalent Streptavidin with a Peroxidase Inhibitor

In this example the presence of endogenous peroxidase was also scored as well as the presence of endogenous biotin. The endogenous peroxidase activity was mostly present in the form of staining of red blood cells (RBC's).

Experimental Procedure:

Two different peroxidase inhibitors were prepared. The first peroxidase inhibitor contained 0.1% Hydrogen peroxide (H₂O₂) in methanol (MeOH). The second peroxidase inhibitor contained 0.1% H₂O₂ and 0.1% Sodium aside (NaN₃). In some instances, the peroxidase inhibitors contained high-affinity monovalent streptavidin.
Step 1: Tissues were incubated with the peroxidase inhibitor for five minutes.
Step 2: Tissues were rinsed and then incubated with a primary antibody to CK for 10 minutes.
Step 3: Tissues were rinsed and then incubated for 10 minutes with a biotinylated secondary antibody of goat-antimouse immunoglobulin.
Step 4: Tissues were rinsed and then incubated for 10 minutes with a solution of SA-FWIR in TB S at a concentration of 10.0 ug/ml.
Step 5: Tissues were rinsed and then incubated for five minutes with a solution of DAB.

TABLE 8
Example 7 Group Treatment

| | | Primary | Biotinylated | | |
|---|---|---|---|---|---|
| Group | Peroxidase Inhibitor | AB CK | 2nd Ab | SA-HRP | DAB |
| 1 | No | No | No | No | Yes |
| 2 | No | No | No | Yes | Yes |
| 3 | H₂O₂ + MeOH | No | No | Yes | Yes |
| 4 | H₂O₂ + NaN₃ | No | No | Yes | Yes |
| 5 | H₂O₂ + MeOH + | No | No | Yes | Yes |
| | monomeric SA | | | | |
| 6 | H₂O₂ + NaN₃ + | No | No | Yes | Yes |
| | monomeric SA | | | | |
| 7 | H₂O₂+ MeOH + | Yes | Yes | Yes | Yes |
| | monomeric SA | | | | |
| 8 | H₂O₂ + NaN₃ + | Yes | Yes | Yes | Yes |
| | monomeric SA | | | | |

TABLE 9
Example 7 Results

| Group | Staining | Interpretation |
|---|---|---|
| 1 | Yes | Endogenous Peroxidase |
| 2 | Yes | Endogenous Peroxidase and endogenous biotin |
| 3 | Yes | Endogenous biotin only |
| 4 | Yes | Endogenous biotin only |
| 5 | No | Inactivation of endogenous peroxidase and biotin |
| 6 | No | Inactivation of endogenous peroxidase and biotin |
| 7 | Yes | Specific staining CK, Inactivation of endogenous |
| | | peroxidase and biotin |
| 8 | Yes | Specific staining CK, Inactivation of endogenous |
| | | peroxidase and biotin |

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A biotin blocking method, the method comprising the steps of:
reacting a tissue sample with a reagent containing a high-affinity monomeric biotin-binding protein;
reacting the tissue sample with a primary antibody to an analyte under investigation;
reacting the tissue sample with a secondary antibody containing biotin;
reacting the tissue sample with a biotin-binding protein that is linked with an enzyme; and
reacting the tissue sample with a substrate/chromogen reagent to the enzyme.

2. The method of claim 1, wherein the enzyme is selected from the group consisting of an alkaline phosphatase and a peroxidase.

3. The method of claim 1, wherein the tissue sample is simultaneously reacted with the reagent containing a high-affinity monomeric biotin-binding protein and with a peroxidase-blocking reagent.

4. The method of claim 1, wherein the high-affinity monomeric biotin-binding protein comprises a high-affinity monovalent avidin.

5. The method of claim 4, wherein the high-affinity monomeric biotin-binding protein comprises a biotin dissociation constant (KD) that is less than 10.0 Nanomolar (nM).

6. The method of claim 4, wherein the high-affinity monomeric biotin-binding protein comprises a biotin rate constant of dissociation (Koff) that is less than 10.0×10⁻³ minutes⁻¹.

7. The method of claim 1, wherein the high-affinity monomeric biotin-binding protein comprises a high-affinity monovalent streptavidin.

8. The method of claim 7, wherein the high-affinity monomeric biotin-binding protein comprises a biotin dissociation constant (KD) that is less than 10.0 nM.

9. The method of claim 7, wherein the high-affinity monomeric biotin-binding protein comprises a biotin rate constant of dissociation (Koff) that is less than 10.0×10⁻³ minutes⁻¹.

10. The method of claim 1, wherein the high-affinity monomeric biotin-binding protein comprises an avidin family high-affinity monovalent biotin binding protein.

11. The method of claim 10, wherein the high-affinity monomeric biotin-binding protein comprises a biotin dissociation constant (KD) that is less than 10.0 nM.

12. The method of claim 10, wherein the high-affinity monomeric biotin-binding protein comprises a biotin rate constant of dissociation (Koff) that is less than 10.0×10⁻³ minutes⁻¹.

13. A biotin blocking method, the method comprising the steps of:
reacting a tissue sample simultaneously with a reagent containing a high-affinity monomeric biotin-binding protein and with a primary antibody to an analyte under investigation;
reacting the tissue sample with a secondary antibody containing biotin;
reacting the tissue sample with a biotin-binding protein that is linked with an enzyme; and
reacting the tissue sample with a substrate/chromogen reagent to the enzyme.

14. The method of claim 13, wherein the high-affinity monomeric biotin-binding protein comprises a high-affinity monovalent avidin.

15. The method of claim 14, wherein the high-affinity monomeric biotin-binding protein comprises a biotin dissociation constant (KD) that is less than 10.0 Nanomolar (nM).

16. The method of claim 14, wherein the high-affinity monomeric biotin-binding protein comprises a biotin rate constant of dissociation (Koff) that is less than 10.0×10⁻³ minutes⁻¹.

17. The method of claim 13, wherein the high-affinity monomeric biotin-binding protein comprises a high-affinity monovalent streptavidin.

18. The method of claim 17, wherein the high-affinity monomeric biotin-binding protein comprises a biotin dissociation constant (KD) that is less than 10.0 nM.

19. The method of claim 13, wherein the high-affinity monomeric biotin-binding protein comprises an avidin family high-affinity monovalent biotin binding protein.

20. The method of claim 19, wherein the high-affinity monomeric biotin-binding protein comprises a biotin dissociation constant (KD) that is less than 10.0 nM.
